(19) United States
(12) Patent Application Publication
(10) Pub. No.: US 2024/0316203 A1
(43) Pub. Date: Sep. 26, 2024

(54) MEDICAMENT FOR KILLING TUMOR CELLS

(71) Applicant: PhotoQ3 Inc., Tokyo (JP)

(72) Inventors: Takao HAMAKUBO, Tokyo (JP); Naoko TODA, Tokyo (JP); Yukio SUDO, Tokyo (JP); Hiromi KATAOKA, Aichi (JP); Shigenobu YANO, Nara (JP)

(73) Assignee: PhotoQ3 Inc., Tokyo (JP)

(21) Appl. No.: 18/694,587

(22) PCT Filed: Sep. 22, 2022
(86) PCT No.: PCT/JP2022/035381

§ 371 Date: Mar. 22, 2024

(30) Foreign Application Priority Data
Sep. 24, 2021 (JP) 2021-155010

(51) Int. Cl. A61K 41/00; A61K 47/68; A61P 35/00

(52) U.S. Cl. CPC A61K 41/0071; A61K 47/6825; A61K 47/6829; A61K 47/6851; A61P 35/00

(57) ABSTRACT

It is an object of the present invention to provide a medicament for killing tumor cells, having few side effects. According to the present invention, provided is a medicament for killing tumor cells, comprising: a conjugate of a substance that binds to a target substance on the surface of tumor cells and a cytotoxin; and a glycosylated chlorin derivative or a pharmaceutically acceptable salt thereof.

### TECHNICAL FIELD

The present invention relates to a medicament for killing tumor cells, comprising a conjugate of a substance that binds to a target substance on the surface of tumor cells and a cytotoxin, and a glycosylated chlorin derivative or a pharmaceutically acceptable salt thereof.

### BACKGROUND ART

In Japan, approximately 370,000 people die of cancer each year, and cancer has continuously been a leading cause of death since 1981. To date, a large number of therapeutic methods for cancer have been developed inside and outside of the country. However, a specific remedy for cancer has not yet been developed.

What is called, small molecule anticancer agents (cancer chemotherapy) have been developed. However, the medicinal effects of these anticancer agents are not strong enough, and further, severe side effects cause trouble to patients. Thus, it cannot be said that these anticancer agents are desirable medicaments. Since antibody drugs are characterized in that the antibody drugs have strong specificity to cancer, strong side effects found in the small molecule anticancer agents can be alleviated, and thus, such antibody drugs can be broadly used. However, there are yet only a small number of antibody drugs effective for solid cancer. In order to reinforce medicinal effects, ADC (antibody-drug conjugate) has been developed, but such ADC has not yet been satisfactory, including its toxicity.

Moreover, an immunotherapeutic antibody (checkpoint inhibitor) used as a novel antibody drug exhibits strong medicinal effects against a wide range of cancer species based on its novel mechanism. However, it has been found that the number of patients who receive the effects of such an immunotherapeutic antibody is not necessarily large, and that, in some cases, the patients have severe side effects to such an extent that they lead to death.

On the other hand, on the basis of an idea that the therapeutic site is limited and side effects are thereby reduced, PDT (Photo Dynamic Therapy) has been developed. PDT is a therapeutic method by which a light having a certain wavelength that activates photosensitizing dyes gathering in a tumor site is applied to an affected area to treat it. PDT shows certain effects against lung cancer, etc., but it cannot be said that satisfactory medicinal effects are obtained from this therapeutic method.

### SUMMARY OF INVENTION

### Objects to be Solved by the Invention

Under such circumstances, the present inventors have felt a need to develop a pharmaceutical product having few side effects and strong medicinal effects, and thereby achieving the present development goals. It is an object of the present invention to provide a medicament for killing tumor cells, having few side effects.

### Means for Solving the Objects

PDT is a method for destroying tumor cells by accumulating sensitizing dyes to tumor tissues and then applying a light to the tumor cells. As such sensitizing dyes used in PDT, sensitizing dyes having high water-solubility and a high property of accumulating in tumor, such as Talaporfin Sodium, Porfimer Sodium or Verteporfin, have been known, and all of these sensitizing dyes have already been used in the treatment of lung cancer and the like. PDT is a low-invasive therapeutic method, but this method is disadvantageous in that the medicinal effects thereof are not necessarily satisfactory.

PCI (Photochemical Internalization) is a method for destroying an endosomal membrane by accumulating photosensitizers to an endosomal membrane and then applying a light to the endosomal membrane. It is considered that, according to PCI, anticancer agents or immunotoxins enclosed in the endosomal membrane are released into the cytoplasm, so that tumor cells are destroyed. For the purpose of accumulating photosensitizers to the endosomal membrane, the dyes used in PCI are different from those used in PDT, and amphipathic photosensitizers such as sulfonated tetraphenylchlorin (TPCS2a) or aluminum phthalocyanine (AlPcS2a) are used.

However, such amphipathic sulfonated tetraphenylchlorin (TPCS2a) or aluminum phthalocyanine (AlPcS2a) is disadvantageous in that, for example, (1) neurotoxicity is concerned, in that (2) the photosensitizers accumulate in cell membranes other than the endosomal membrane and cause cytotoxicity, and in that (3) the photosensitizers have a low property of accumulating in tumor and cause non-specific side effects.

The present inventors have conducted intensive studies directed towards solving the previous problems. As a result, the present inventors have found that a glycosylated chlorin derivative or a pharmaceutically acceptable salt thereof has such unpredictable effects that it increases the permeability of the endosomal membrane even at a low concentration. Moreover, the present inventors have also found that, by combining a conjugate of a substance that binds to a target substance on the surface of tumor cells and a cytotoxin, with a glycosylated chlorin derivative or a pharmaceutically acceptable salt thereof, cytotoxicity and tumor specificity can be significantly reinforced, thereby completing the present invention.

According to the present invention, the following inventions are provided.

<1> A medicament for killing tumor cells, comprising:
a conjugate of a substance that binds to a target substance on the surface of tumor cells and a cytotoxin; and a glycosylated chlorin derivative or a pharmaceutically acceptable salt thereof.

<2> The medicament according to <1>, wherein the substance that binds to a target substance on the surface of tumor cells is an antibody, an antibody fragment, a ligand, or a peptide.

<3> The medicament according to <1>or <2>, wherein the cytotoxin is saporin, gelonin, or Pseudomonas exotoxin.

<4> The medicament according to any one of <1>to <3>, wherein the tumor cells are cells that express Epidermal Growth Factor Receptor (EGFR, ERBB1, ERBB2, ERBB3, or ERBB4), Mesothelin, Ephrin type-A receptor 2 (EphA2), Glypican 3 (GPC3), Cadherin 17 (CDH17), or Roundabout homolog 1 (Robo1) on the cell surface thereof.

<5> The medicament according to any one of <1>to <4>, wherein the tumor cells are cancer cells of any one of head and neck cancer, lung cancer, liver cancer, colorectal cancer, skin cancer, esophageal cancer, stomach cancer, cervical cancer, endometrial cancer, mesothelioma, brain tumor, malignant melanoma, breast cancer, bile duct cancer, pancreatic cancer, ovarian cancer, kidney cancer, bladder cancer, prostate cancer, malignant lymphoma, and osteosarcoma.

<6> The medicament according to any one of <1>to <5>, which kills tumor cells by performing the following steps on the cells:
(1) a step of allowing the conjugate to come into contact with tumor cells:
(2) a step of allowing a glycosylated chlorin derivative or a pharmaceutically acceptable salt thereof to come into contact with the tumor cells; and
(3) a step of irradiating the tumor cells with a wavelength effective for activating the glycosylated chlorin derivative or a pharmaceutically acceptable salt thereof, so as to kill the cells.

<7> The medicament according to any one of <1>to <5>, which kills tumor cells by performing the following steps on the cells:
(1) a step of allowing a glycosylated chlorin derivative or a pharmaceutically acceptable salt thereof to come into contact with tumor cells
(2) a step of allowing the conjugate to come into contact with the tumor cells: and
(3) a step of irradiating the tumor cells with a wavelength effective for activating the glycosylated chlorin derivative or a pharmaceutically acceptable salt thereof, so as to kill the cells.

<8> The medicament according to any one of <1>to <5>, which kills tumor cells by performing the following steps on the cells:
(1) a step of allowing the conjugate, and a glycosylated chlorin derivative or a pharmaceutically acceptable salt thereof, to come into contact with tumor cells; and then,
(2) a step of irradiating the tumor cells with a wavelength effective for activating the glycosylated chlorin derivative or a pharmaceutically acceptable salt thereof, so as to kill the cells.

<9> The medicament according to any one of <6>to <8>, wherein the wavelength effective for activating the glycosylated chlorin derivative or a pharmaceutically acceptable salt thereof is 600 to 800 nm.

<A> A method for killing tumor cells, comprising:
(1) a step of allowing a conjugate of a substance that binds to a target substance on the surface of tumor cells and a cytotoxin to come into contact with tumor cells:
(2) a step of allowing a glycosylated chlorin derivative or a pharmaceutically acceptable salt thereof to come into contact with the tumor cells: and
(3) a step of irradiating the tumor cells with a wavelength effective for activating the glycosylated chlorin derivative or a pharmaceutically acceptable salt thereof, so as to kill the cells.

<B> A method for killing tumor cells, comprising:
(1) a step of allowing a conjugate of a substance that binds to a target substance on the surface of tumor cells and a cytotoxin, and a glycosylated chlorin derivative or a pharmaceutically acceptable salt thereof, to come into contact with tumor cells; and then
(2) a step of irradiating the tumor cells with a wavelength effective for activating the glycosylated chlorin derivative or a pharmaceutically acceptable salt thereof, so as to kill the cells.

### Advantageous Effects of Invention

According to the present invention, a medicament for killing tumor cells, having few side effects, can be provided.

### BRIEF DESCRIPTION OF DRAWINGS

FIG. 1 shows the results of a cytotoxicity assay, in which an EGFR-expressing cell line (A431), glycosylated chlorin, and IT-Cetuximab were used.

FIG. 2 shows the results of a cytotoxicity assay, in which an EGFR-expressing cell line (A549), glycosylated chlorin, and IT-Cetuximab were used.

### EMBODIMENT OF CARRYING OUT THE INVENTION

Hereinafter, the embodiments of the present invention will be described in detail.

### Summary of the Invention

The present inventors have studied a method for treating a tumor, which has strong medicinal effects and few side effects, and as a result, they have conceived that the techniques PDT and PCI that topically enhance medicinal effects as a result of light irradiation have potential. However, PDT has been disadvantageous in terms of weak medicinal effects, whereas PCI has not been satisfactory in terms of both medicinal effects and toxicity.

Hence, the present inventors have conducted intensive studies, and as a result, they have found for the first time that a water-soluble sensitizing dye, namely, a glycosylated chlorin derivative or a pharmaceutically acceptable salt thereof improves the permeability of the endosome by light irradiation. By combining this “dye,” “a conjugate of a substance that binds to a target substance on the surface of tumor cells and a cytotoxin,” and “light irradiation to a tumor,” the present inventors have discovered a therapeutic method having strong medicinal effects and few side effects, thereby completing the present invention.

Specifically, in the present invention, a conjugate of a substance that binds to a target substance on the surface of tumor cells and a cytotoxin binds to a tumor, and it is then enclosed in the endosome. It is conceived that a light is then applied to Talaporfin Sodium, Porfimer Sodium or Verteporfin, which has been added separately (or simultaneously), so that an immunotoxin (or a decomposed product thereof) in the endosome is released into the cytoplasm, and thereby, the tumor cells can be killed.

### Embodiments Regarding Method for Killing Cells

The method for killing tumor cells of the present invention may include an embodiment in which tumor cells are irradiated with (3) a light having a wavelength for activating a glycosylated chlorin derivative or a pharmaceutically acceptable salt thereof, in the coexistence of (1) a conjugate of a substance that binds to a target substance on the surface of tumor cells and a cytotoxin, and (2) the glycosylated chlorin derivative or a pharmaceutically acceptable salt thereof.

As a further specific embodiment, the killing of tumor cells can also be achieved by administering (1) a conjugate of a substance that binds to a target substance on the surface of tumor cells and a cytotoxin to a subject, then administering thereto (2) a glycosylated chlorin derivative or a pharmaceutically acceptable salt thereof, and then irradiating the cells with (3) a light having a wavelength for activating the glycosylated chlorin derivative or a pharmaceutically acceptable salt thereof.

In addition, as another embodiment, the killing of tumor cells can also be achieved by administering (1) a glycosylated chlorin derivative or a pharmaceutically acceptable salt thereof to a subject, then administering thereto (2) a conjugate of a substance that binds to a target substance on the surface of tumor cells and a cytotoxin, and then irradiating the cells with (3) a light having a wavelength for activating the glycosylated chlorin derivative or a pharmaceutically acceptable salt thereof.

Moreover, as another embodiment, the killing of tumor cells can also be achieved by simultaneously administering (1) a conjugate of a substance that binds to a target substance on the surface of tumor cells and a cytotoxin and (2) a glycosylated chlorin derivative or a pharmaceutically acceptable salt thereof to a subject, and then irradiating the cells with (3) a light having a wavelength for activating the glycosylated chlorin derivative or a pharmaceutically acceptable salt thereof. The wavelength for activating the glycosylated chlorin derivative or a pharmaceutically acceptable salt thereof is preferably 600 to 800 nm, more preferably 600 to 750 nm, further preferably 600 to 700 nm, and particularly preferably 650 to 680 nm, and the wavelength is, for example, 660 to 662 nm.

### Photosensitizing Dye

In the present invention, a glycosylated chlorin derivative or a pharmaceutically acceptable salt thereof is used as a sensitizer.

As such a glycosylated chlorin derivative, the glycosylated chlorin e6 derivative represented by the following general formula (1) of International Publication WO2018/10143 can be used. The glycosylated chlorin e6 derivative represented by the general formula (1) will be described below.

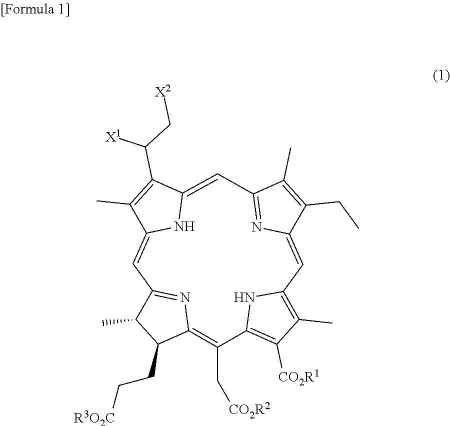

In the general formula (1), X1 and X2 each independently represent H (hydrogen atom) or a group represented by R-X-* (wherein * indicates a binding site), and at least one of X1 and X2 represents a group represented by R-X-*. Either X1 or X2 is preferably the group represented by R—X—* , and more preferably, X1 is the group represented by R—X—* and X2 is H (hydrogen atom).

Herein, R represents the residue of a sugar (hereinafter referred to as a “sugar residue”). Sugar residue indicates a residue, from which one hydroxyl group binding to a carbon atom possessed by a sugar is removed. A residue, from which a hemiacetal (anomeric) hydroxyl group of a sugar is removed, is preferable.

X is a divalent group binding to any one of carbon atoms that constitute R: and R is a linear or branched divalent group, consisting of at least one type of atoms selected from the group consisting of C (carbon atom), N (nitrogen atom), O (oxygen atom), H (hydrogen atom), and S (sulfur atom). Examples of X may include —S—, —O—, —NRx— (wherein Rx represents a hydrogen atom, or a hydrocarbon atom optionally having a heteroatom), a carbonyl group, an alkylene group, an alkenylene group, and a group formed by combining these with one another. X preferably includes O (oxygen atom) and/or S (sulfur atom): X is more preferably a group formed by combining 2 or more types selected from the group consisting of —S—, —O—, and an alkylene group, with one another: and X is further preferably a group formed by combining —S—, —O—, and an alkylene group with one another.

The sugar of R is not particularly limited, and examples thereof may include:
monosaccharides such as aldopentose (ribose, arabinose, xylose, and liquisose, etc.), aldohexose (allose, altrose, glucose, mannose, gulose, idose, galactose, and talose, etc.), aldoheptose, ketopentose (ribulose and xylulose, etc.), ketohexose (psicose, fructose, sorbose, and tagatose, etc.), ketoheptose (sedheptulose and choriose, etc.), and their derivatives having amino groups:
oligosaccharides such as sucrose, maltose, lactose, maltotriose, raffinose and maltotetraose, and their derivatives having amino groups: and
polysaccharides such as starch, amylose and glycogen, and their derivatives having amino groups. Among others, monosaccharides are preferable: hexose or hexosamine is more preferable: hexose is further preferable: and glucose is particularly preferable.

The monosaccharide may be either a D-form or an L-form, and the D-form is preferable.

Besides, in the present description, oligosaccharide means a compound containing 2 to 9 monosaccharide units, and polysaccharide means a compound containing more than 10 monosaccharide units (J. D. ROBERTS & M. C. CASERIO (1964). BASIC PRINCIPLES OF ORGANIC CHEMISTRY. W. A. Benjamin. Inc. (cited from Roberts Organic Chemistry, J. D. ROBERTS & M. C. CASERIO, (translated by) Michinori Ohgi (1969), Tokyo Kagaku Dojin Co., Ltd.). Monosaccharides that bind to each other via a glycoside bond may be the same as or different from each other. In addition, the glycoside bond between monosaccharides may be either an a-bond or a β-bond.

Specific examples of hexose may include glucose, galactose, mannose, allose, altrose, gulose, idose, and, talose. Among these, glucose is most preferable. This is because phototoxicity is excellent when the hexose is glucose.

Specific examples of hexosamine may include glucosamine, galactosamine, mannosamine, daunosamine, and, perosamine. Among these, glucosamine is most preferable. This is because phototoxicity is excellent when the hexosamine is glucosamine.

In the formula (1), R¹, R² and R³ each independently represent H (hydrogen atom), an acetoxyalkyl group containing 1 to 6 carbon atoms, or a hydrocarbon group containing 1 to 6 carbon atoms; and at least one of R¹, R² and R³ is an acetoxyalkyl group containing 1 to 6 carbon atoms or a hydrocarbon group containing 1 to 6 carbon atoms.

Herein, examples of the acetoxyalkyl containing 1 to 6 carbon atoms may include acetoxymethyl, acetoxyethyl, acetoxypropyl, and acetoxy butyl. Examples of the hydrocarbon containing 1 to 6 carbon atoms may include linear, branched or cyclic alkyls containing 1 to 6 carbon atoms, such as methyl, ethyl, n-propyl, n-butyl, sec-butyl, tert-butyl, pentyl, cyclopentyl, hexyl, and cyclohexyl. Among others, it is preferable that R¹, R² and R³ are each independently an acetoxyalkyl group containing 1 to 6 carbon atoms, or a hydrocarbon group containing 1 to 6 carbon atoms.

Moreover, from the viewpoint of water solubility, it is preferable that R¹, R² and R³ are each independently a hydrocarbon group containing 1 to 3 carbon atoms, and it is more preferable that R¹, R² and R³ are each independently a methyl group.

In the group represented by R—X—* (wherein * represents a binding site), the divalent group (linking group) X preferably contains O (oxygen atom), and more preferably contains O and S (sulfur atom).

Among others, the group represented by R—X—* is preferably a group represented by R—X³—O—*. In this formula, X³ is a linear or branched divalent group that consists of at least one selected from the group consisting of C, N, O, H and S, wherein the divalent group binds to any one of carbon atoms constituting R. The divalent group X³ is not particularly limited, and it has the same form as that of the divalent group X that has already been explained above.

That is to say, the glycosylated chlorin e6 derivative is preferably represented by the following formula (2). Besides, in the formula (2), the form of the sugar residue R is as already explained for R in the formula (1).

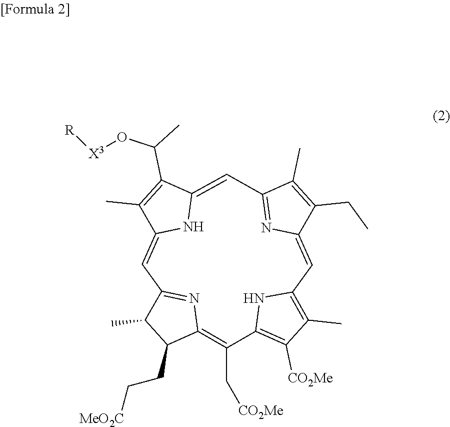

Furthermore, from the viewpoint of obtaining a further excellent glycosylated chlorin e6 derivative having the effects of the present invention, the group represented by R—X³—O—* is more preferably a group represented by R—L—S—X⁴—O—*. In this formula, L represents a single bond or a divalent group. The divalent linking group L is not particularly limited, and it is, for example, as already explained for the divalent group X.

Among others, L in the group represented by R—L—S—X⁴—O—* is preferably a single bond. That is to say, the group represented by R—X—* is preferably a group represented by R—S—X⁴—O—*, and in other words, it is preferably a group in which the sugar residue R is directly linked to —S—X⁴—O—. Herein, the term “direct linkage” refers to a structure (—C—S—X⁴—O—), in which C (carbon atom) at the anomeric position of a sugar is linked to —S—X⁴—O—, for example.

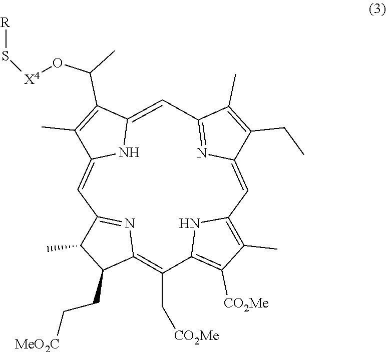

Moreover, from the viewpoint of synthesis, S (sulfur atom) in the group represented by R—S—X⁴—O—* is preferably a linking group that binds to the carbon atom at the anomeric position (the carbon atom at position 1) of R, or a linking group that binds to a carbon atom adjacent to the carbon atom at the anomeric position (the carbon atom at position 2); and is more preferably a linking group that binds to the carbon atom at the anomeric position (the carbon atom at position 1) of R.

X⁴ binds to O (oxygen atom) and S (sulfur atom). In addition, X⁴ is a linear or branched divalent group having C (carbon atom) and H (hydrogen atom). X⁴ is not particularly limited, and examples of X⁴ may include an alkylene group, an oxyalkylene group, and alkyleneoxy group. Among others, X⁴ is preferably a linear or branched alkylene group containing 1 to 16 carbon atoms, and is more preferably a linear alkylene group represented by —(CH₂)n—. Herein, n is preferably an integer of 1 to 16, n is more preferably an integer of 2 to 13, and n is further preferably an integer of 3 to 10.

Besides, the form of the sugar residue R in the formula (3) is as already explained for R in the formula (1).

Among others, the sugar residue R in the formula (3) is preferably a compound represented by the following formula (4), (5), or (6). In the following formulae (4) to (6), n represents an integer of 3 to 10. respectively.

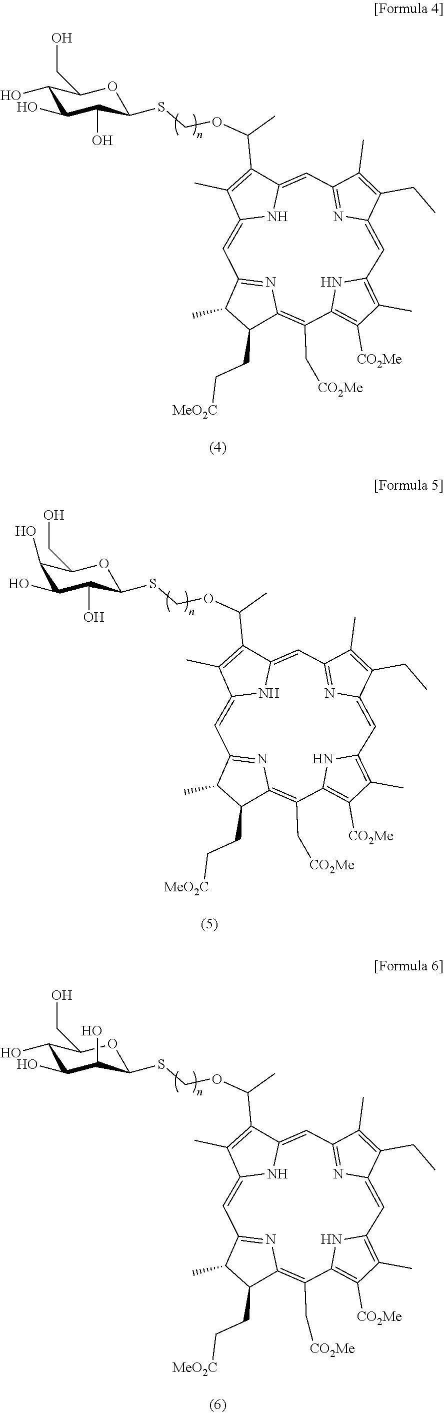

Examples of the pharmaceutically acceptable salt may include alkali metal salts (e.g. sodium salts and potassium salts, etc.), alkaline earth metal salts (e.g. magnesium salts and calcium salts, etc.), ammonium salts, mono-, di-or tri-lower (alkyl or hydroxyalkyl)ammonium salts (e.g. ethanol ammonium salts, diethanol ammonium salts, triethanolamine salts, and tromethamine salts, etc.), hydrochloride, hydrobromate, hydroiodate, nitrate, phosphate, sulfate, formate, acetate, citrate, oxalate, fumarate, maleate, succinate, malate, tartrate, trichloroacetate, trifluoroacetate, methanesulfonate, benzenesulfonate, p-toluenesulfonate, mesitylenesulfonate, and naphthalenesulfonate. In addition, the salt may be an anhydride or a solvate. Examples of the solvate may include a hydrate, a methanol solvate, an ethanol solvate, a propanol solvate, and a 2-propanol solvate.

### Substance that Binds to Target Substance on Surface of Tumor Cells

Examples of the substance that binds to a target substance on the surface of tumor cells may include, but are not particularly limited to, an antibody, an antibody fragment, a ligand, and a peptide.

When an antibody is used as such a substance that binds to a target substance on the surface of tumor cells, it is possible to use an antibody that specifically binds to a target substance on the surface of tumor cells (for example, a protein, such as Epidermal Growth Factor Receptor (EGFR, ERBB1, ERBB2, ERBB3, or ERBB4), Mesothelin, Ephrin type-A receptor 2 (EphA2), Glypican 3 (GPC3), Cadhelin 17 (CDH17), Cadherin 3 (CDH3), or Roundabout homolog 1 (Robo1)).

The type of the antibody used in the present invention is not particularly limited, and examples of the present antibody may include a mouse antibody, a human antibody, a rat antibody, a rabbit antibody, a sheep antibody, a camel antibody, an avian antibody, and a genetically modified antibody that is artificially modified for the purpose of reducing xenoantigenicity against a human, such as a chimeric antibody or a humanized antibody. Such a genetically modified antibody can be produced by applying a known method. The chimeric antibody is an antibody consisting of the heavy chain and light chain variable regions of a mammalian antibody other than a human antibody, such as a mouse antibody, and the heavy chain and light chain constant regions of a human antibody. The chimeric antibody can be obtained by ligating DNA encoding the variable region of a mouse antibody to DNA encoding the constant region of a human antibody, then incorporating the ligate into an expression vector, and then introducing the expression vector into a host, so that the host is allowed to generate the antibody. The humanized antibody is obtained by transplanting the complementarity determining region (CDR) of a mammalian antibody other than a human antibody, such as a mouse antibody, into the complementarity determining region of a human antibody. A common gene recombination method therefor has been known. Specifically, a DNA sequence designed to ligate the CDR of a mouse antibody to the framework region (FR) of a human antibody is synthesized from several oligonucleotides that have been produced such that they have an overlapping portion at the terminal portions thereof according to a PCR method. The obtained DNA is ligated to DNA encoding the constant region of a human antibody, and the ligate is then incorporated into an expression vector, which is then introduced into a host, so that the host is allowed to generate the antibody (EP 239400, International Publication WO96/02576, etc.).

In addition, a method of obtaining a human antibody has also been known. For example, human lymphocytes are sensitized with a desired antigen or a cell expressing the desired antigen in vitro, and then fusing the sensitized lymphocytes with human myeloma cells, such as, for example, U266, so as to obtain a desired human antibody having a binding activity to an antigen (JP Paten Publication (Kokoku) No. 1-59878 B (1989)). Otherwise, a transgenic antibody having all repertoires of human antibody genes is immunized with a desired antigen to obtain a desired human antibody (see WO93/12227, WO92/03918, WO94/02602, WO94/25585, WO96/34096, and WO96/33735). Further, a technique of obtaining a human antibody by panning using a human antibody library has also been known. For example, a human antibody variable region is allowed to express as a single chain antibody (scFv) on the surface of a phage according to a phage display method, and a phage binding to an antigen can be then selected. By analyzing the selected phage gene, a DNA sequence encoding the variable region of a human antibody binding to the antigen can be determined. If the DNA sequence of scFv binding to an antigen is clarified, a suitable expression vector comprising the sequence can be produced, so that a human antibody can be obtained. These methods have already been publicly known, and please refer to WO92/01047, WO92/20791, WO93/06213, WO93/11236, WO93/19172, WO95/01438, and WO95/15388.

The antibody that binds to tumor cells is preferably a humanized or a human antibody, but is not limited thereto.

Moreover, these antibodies may also be low molecular weight antibodies such as antibody fragments, or modified forms of the antibodies, unless they lose the property of recognizing the entire or a part of a protein encoded by an antigen gene present on the surface of tumor cells. The antibody fragment is a part of an antibody that retains a binding ability to ROBO1. Specific examples of the antibody fragment may include Fab, Fab′, F(ab′)2, Fv, Diabody, and a single chain variable fragment (scFv). In order to obtain such an antibody fragment, a gene encoding such an antibody fragment is constructed, the gene is then introduced into an expression vector, and it may be then expressed in suitable host cells. As a modified form of an antibody, an antibody binding to various types of molecules such as polyethylene glycol (PEG) can also be used.

DNA encoding a monoclonal antibody can be easily isolated and sequenced according to a commonly used method (for example, by using an oligonucleotide probe capable of specifically binding to a gene encoding the heavy chain and light chain of the monoclonal antibody). Hybridoma cells may be preferable starting materials for such DNA. Once such DNA is isolated, it is inserted into an expression vector, and the expression vector is then used to transform host cells such as E. coli cells, COS cells, CHO cells, or myeloma cells that do not generate immunoglobulin before they are transformed. Then, a monoclonal antibody can be generated from the transformed host cells.

As a substance that binds to a target substance on the surface of tumor cells, a ligand can be used. When the target substance on the surface of tumor cells is, for example, a receptor such as Epidermal Growth Factor Receptor (EGFR, ERBB1, ERBB2, ERBB3, or ERBB4), Mesothelin, or Ephrin type-A receptor 2 (EphA2), a ligand against each of the above-described receptors can be used.

As such a substance that binds to a target substance on the surface of tumor cells, a peptide can also be used. A peptide that binds to a target substance on the surface of tumor cells can be designed and produced by those skilled in the art.

### Cytotoxin

The cytotoxin is preferably a protein having cytotoxicity, but is not limited thereto. The cytotoxin may also be a compound having a synthetic or natural anticancer action, such as bleomycin, or a compound used in ADC.

Preferred examples of such a protein having cytotoxicity may include saporin, gelonin, Pseudomonas exotoxin, ricin A chain, deglycosylated ricin A chain, a ribosome inactivating protein, alphasarcine, aspergillin, restrictocin, ribonuclease, epipodophyllotoxin, diphtheria toxin, Shigatoxin, and a mutant or a genetically modified body thereof.

### Conjugate of Substance that Binds to Target Substance on Surface of Tumor Cells and Cytotoxin

A substance that binds to a target substance on the surface of tumor cells, and a cytotoxin, must bind to each other, directly or indirectly.

When an antibody or a fragment thereof is used as such a substance that binds to a target substance on the surface of tumor cells, as a method of directly chemically binding the antibody or the fragment thereof to a cytotoxin, a binding method used for known ADC (Antibody Drug Conjugate) can be used. Otherwise, when the cytotoxin is a protein, a bifunctional crosslinking agent can also be used.

Alternatively, when the cytotoxin is a protein, a toxin is fused with an antibody or a fragment thereof by genetic recombination to form a protein, so that an immunotoxin can be produced.

Moreover, as another method, a method of indirectly binding an antibody or a fragment thereof to a cytotoxin by using a second binding pair can also be used. Examples of the second binding pair that can be utilized herein may include avidin-biotin and an antibody-hapten.

Further, in the present invention, it is also possible to use a conjugate of a peptide or a ligand that binds to a target substance on the surface of tumor cells and a toxin, instead of using an immunotoxin in which an antibody and a toxin bind to each other.

### Administration Methods and Applied Doses

The method for administering the medicament of the present invention to a subject having a tumor (for example, a cancer, etc.) is not particularly limited.

The conjugate of a substance that binds to a target substance on the surface of tumor cells and a cytotoxin can be administered to the subject, for example, via intravenous administration, arterial administration, intramuscular administration, subcutaneous administration, intradermal administration, intraperitoneal administration, or oral administration. Alternatively, an administration method involving administration of the conjugate to tumor tissues and the periphery thereof via local injection, application, spraying or the like can also be applied.

A glycosylated chlorin derivative or a pharmaceutically acceptable salt thereof can be administered to a subject, for example, via intravenous administration, arterial administration, intramuscular administration, subcutaneous administration, intradermal administration, intraperitoneal administration, or oral administration. Alternatively, an administration method involving administration of the glycosylated chlorin derivative or a pharmaceutically acceptable salt thereof to tumor tissues and the periphery thereof via local injection, application, spraying or the like can also be applied.

The applied dose of the conjugate of a substance that binds to a target substance on the surface of tumor cells and a cytotoxin is not particularly limited. The conjugate can be administered to a subject at a dose of, for example, 1 μg/kg of body weight to 100 mg/kg of body weight, and preferably, at a dose of 10 μg/kg of body weight to 10 mg/kg of body weight.

The applied dose of the glycosylated chlorin derivative or a pharmaceutically acceptable salt thereof is not particularly limited. The glycosylated chlorin derivative or a pharmaceutically acceptable salt thereof can be administered to a subject at a dose of, for example, 1 μg/kg of body weight to 100 mg/kg of body weight, and preferably, at a dose of 10 μg/kg of body weight to 10 mg/kg of body weight.

The number of doses is not particularly limited, and administration can be carried out once to several times (from once to 20 times, and preferably from once to 10 times). Administration can be carried out, for example, every 2 to 4 weeks, or every 1 to 2 months. In addition, the number of light irradiation operations is not particularly limited, either. The light irradiation can be carried out once to several times.

### Cells and/or Diseases as Targets

The tumor as a target of the administration of the medicament of the present invention is a tumor that expresses Epidermal Growth Factor Receptor (EGFR, ERBB1, ERBB2, ERBB3, or ERBB4), Mesothelin, Ephrin type-A receptor 2 (EphA2), Glypican 3 (GPC3), Cadhelin 17 (CDH17), Cadhelin 3 (CDH3), Roundabout homolog 1 (Robo1) or the like on the surface thereof.

Specific examples of the tumor as a target of the administration of the medicament of the present invention may include cancers, such as head and neck cancer, lung cancer, liver cancer, colorectal cancer, skin cancer, esophageal cancer, stomach cancer, cervical cancer, endometrial cancer, mesothelioma, brain tumor, malignant melanoma, breast cancer, bile duct cancer, pancreatic cancer, ovarian cancer, kidney cancer, bladder cancer, prostate cancer, malignant lymphoma, and osteosarcoma.

Moreover, the present invention can be used in the treatment of animals other than humans, such as dogs, cats or horses, as well as the treatment of the diseases of humans.

### EXAMPLES

In the following Examples, the 1-(3-hydroxy-propanethio)-β-D-glucose-conjugated chlorin e6 trimethyl ester described in [Example 1] of International Publication WO2018/10143 was used as a sugar chain-conjugated chlorin (i.e. a glycosylated chlorin derivative) serving as a photosensitizer.

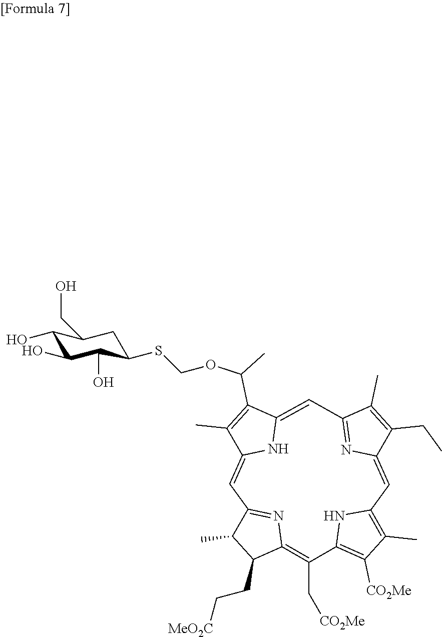

### Example 1: Cytotoxicity Assay Using EGFR-Expressing Cell Line (A431), Sugar Chain-Conjugated Chlorin, and IT-Cetuximab

### <Acquisition of Materials>

As an EGFR-expressing cell line, A431 (human epithelial-like cell carcinoma-derived cell line) was acquired from KAC Company (Kyoto, Japan). As an anti-EGFR antibody, Cetuximab was acquired from Selleck Biotech, Ltd. (Tokyo, Japan). An LED lamp (54 W) having a peak wavelength at 650 nm was purchased from King Do Way (18PCS E27, Amazon.co.jp).

### <Cell Culture>

Using a medium prepared by adding 10% fetal bovine serum to a high-glucose Dulbecco's Modified Eagle's Medium (DMEM), A431 was cultured under conditions of 37° C. and 5% CO₂ concentration.

### <Adjustment of Immunotoxin>

Cetuximab dissolved in PBS(−) was mixed with EZ-LINK sulfo-NHS-LC-biotinylation reagent (Thermo Fisher Scientific, Commonwealth of Massachusetts) dissolved in ultrapure water at a molar ratio of 1:40, and the obtained mixture was then reacted. Thereafter, the reaction mixture was purified using PD SpinTrap G-25 (GE Healthcare Life Sciences, England). The biotinylated Cetuximab was equivalently mixed with streptavidin-saporin (Biotin-Z Internalization Kit [KIT-27-Z], Advanced Targeting Systems, California), and the obtained mixture was reacted at room temperature for 30 minutes, so as to obtain saporin-conjugated Cetuximab (IT-Cetuximab).

### <Cytotoxicity Assay>

A431 was seeded in a 96-well plate in an amount of 1.0×10⁴ cells per well, and was then cultured overnight. Subsequently, under the following three conditions, addition of an immunotoxin and a photosensitizer and light irradiation were carried out, and the degrees of cytotoxicity under individual conditions were compared with one another.
Condition 1: Addition of only 0.006-4 nM IT-Cetuximab
Condition 2: Addition of 0.006-4 nM IT-Cetuximab and 30 nM sugar chain-conjugated chlorin
Condition 3: Light irradiation (650 nm, 18.8 mJ/cm2), after addition of 0.006-4 nM IT-Cetuximab and 30 nM sugar chain-conjugated chlorin

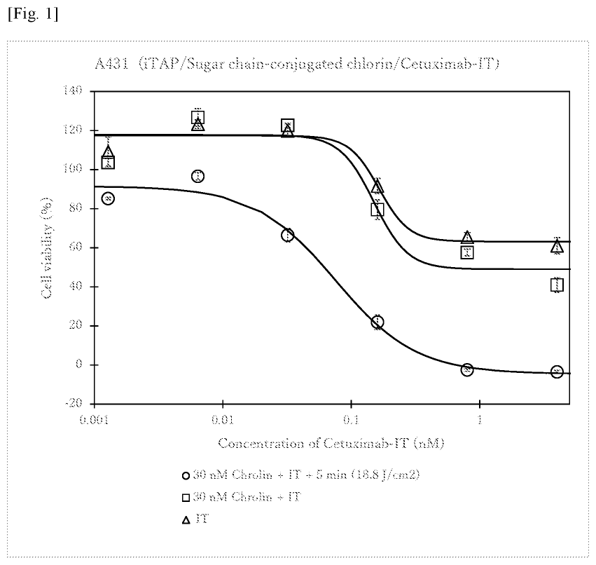

IT-Cetuximab was added to the cells, and 20 hours later, sugar chain-conjugated chlorin was added to the cells under individual conditions. Further 4 hours later, the cells were washed once using PBS(−), and a novel drug-free medium was then added to the resulting cells. The cell group under Condition 3 was irradiated with a light of 650 nm, so as to result in 18.8 J/cm². After the cells had been cultured for 72 hours, CCK-8 kit solution (Cell Counting Kit-8, DOJINDO LABORATORIES, Japan) was added in an amount of 10 μl to each well, and the reaction was then carried out for 1 to 2 hours. Thereafter, the absorption at 450 nm of each well was measured. Using the obtained results, cell viability was calculated according to the following calculating equation (FIG. 1):

Cell viability (%)=(a−c)/(b−c)×100.

In the above equation, “a” indicates the absorbance of each specimen, “b” indicates the absorbance of a negative control specimen not containing IT, and “c” indicates the absorbance of a medium alone. Regarding the EC50 value, using the open source analysis software ImageJ, the mean results were fitted to a sigmoid curve, so as to obtain the IT-Cetuximab concentration showing 50% viability.

### <Results>

In the cell group in which IT-Cetuximab and sugar chain-conjugated chlorin were simultaneously added to the A431 cell line, an IT-Cetuximab concentration-dependent cytotoxic activity was observed, and the 50% effective concentration (EC50) was found to be 0.54 nM. On the other hand, in the cell group under Condition 3, in which IT-Cetuximab and Laserphyrin were simultaneously added to the cell line, followed by light irradiation, the cytotoxic activity was significantly increased, and the EC50 value was 0.06 nM.

### Example 2: Cytotoxicity Assay Using EGFR-Expressing cell line (A549), Sugar Chain-Conjugated Chlorin, and IT-Cetuximab

### <Acquisition of Materials>

As an EGFR-expressing cell line, A549 (human lung cancer cells) was acquired from KAC Company (Kyoto, Japan). The same Cetuximab serving as an anti-EGFR antibody and the same LED lamp having a peak wavelength at 650 nm, as those used in Example 1, were used in the present example.

### <Cell Culture>

Using a medium prepared by adding 10% fetal bovine serum to a high-glucose Dulbecco's Modified Eagle's Medium (DMEM), A549 was cultured under conditions of 37° C. and 5% CO₂ concentration.

### <Adjustment of Immunotoxin>

The same saporin-conjugated Cetuximab (IT-Cetuximab) as that produced in Example 1 was used herein.

### <Cytotoxicity Assay>

A549 was seeded in a 96-well plate in an amount of 2.5×10³ cells per well, and was then cultured overnight. Subsequently, under the following three conditions, addition of an immunotoxin and a photosensitizer and light irradiation were carried out, and the degrees of cytotoxicity under individual conditions were compared with one another.
Condition 1: Addition of only 0.006-4 nM IT-Cetuximab
Condition 2: Addition of 0.006-4 nM IT-Cetuximab and 100 nM sugar chain-conjugated chlorin
Condition 3: Light irradiation (650 nm, 18.8 mJ/cm²), after addition of 0.006-4 nM IT-Cetuximab and 100 nM sugar chain-conjugated chlorin

IT-Cetuximab was added to the cells, and 20 hours later, sugar chain-conjugated chlorin was added to the cells under individual conditions. Further 4 hours later, the cells were washed once using PBS(−), and a novel drug-free medium was then added to the resulting cells. The cell group under Condition 3 was irradiated with a light of 650 nm, so as to result in 18.8 J/cm².

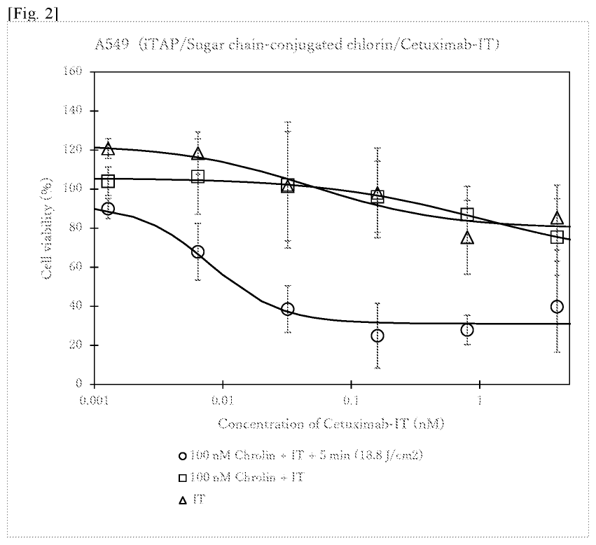

After the cells had been cultured for 72 hours, the CCK-8 kit solution was added in an amount of 10 μl to each well, and the reaction was then carried out for 1 to 2 hours. Thereafter, the absorption at 450 nm of each well was measured. Based on the absorbance obtained in the same manner as that of Example 1, the cell viability (FIG. 2) and the IT-Cetuximab concentration showing 50% viability were calculated.

### <Results>

IT-Cetuximab was confirmed to have almost no concentration-dependent cytotoxic activity against the A549 cell line. On the other hand, in the cell group under Condition 3. in which IT-Cetuximab and sugar chain-conjugated chlorin were added to the cell line. followed by light irradiation. the cytotoxic activity was significantly increased. and the EC50 value was 0.013 nM.

1. A medicament for killing tumor cells, comprising:
a conjugate of a substance that binds to a target substance on the surface of tumor cells and a cytotoxin; and
a glycosylated chlorin derivative or a pharmaceutically acceptable salt thereof.

2. The medicament according to claim 1, wherein the substance that binds to a target substance on the surface of tumor cells is an antibody, an antibody fragment, a ligand, or a peptide.

3. The medicament according to claim 1, wherein the cytotoxin is saporin, gelonin, or Pseudomonas exotoxin.

4. The medicament according to claim 1, wherein the tumor cells are cells that express Epidermal Growth Factor Receptor (EGFR, ERBB1, ERBB2, ERBB3, or ERBB4), Mesothelin, Ephrin type-A receptor 2 (EphA2), Glypican 3 (GPC3), Cadherin 17 (CDH17), or Roundabout homolog 1 (Robo1) on the cell surface thereof.

5. The medicament according to claim 1, wherein the tumor cells are cancer cells of any one of head and neck cancer, lung cancer, liver cancer, colorectal cancer, skin cancer, esophageal cancer, stomach cancer, cervical cancer, endometrial cancer, mesothelioma, brain tumor, malignant melanoma, breast cancer, bile duct cancer, pancreatic cancer, ovarian cancer, kidney cancer, bladder cancer, prostate cancer, malignant lymphoma, and osteosarcoma.

6. The medicament according to claim 1, which kills tumor cells by performing the following on the cells:
(1) allowing the conjugate to come into contact with tumor cells;
(2) of allowing a glycosylated chlorin derivative or a pharmaceutically acceptable salt thereof to come into contact with the tumor cells; and
(3) irradiating the tumor cells with a wavelength effective for activating the glycosylated chlorin derivative or a pharmaceutically acceptable salt thereof, so as to kill the cells.

7. The medicament according to claim 1, which kills tumor cells by performing the following on the cells:
(1) of allowing a glycosylated chlorin derivative or a pharmaceutically acceptable salt thereof to come into contact with tumor cells
(2) allowing the conjugate to come into contact with the tumor cells; and
(3) irradiating the tumor cells with a wavelength effective for activating the glycosylated chlorin derivative or a pharmaceutically acceptable salt thereof, so as to kill the cells.

8. The medicament according to claim 1, which kills tumor cells by performing the following on the cells:
(1) allowing the conjugate, and a glycosylated chlorin derivative or a pharmaceutically acceptable salt thereof, to come into contact with tumor cells; and then,
(2) irradiating the tumor cells with a wavelength effective for activating the glycosylated chlorin derivative or a pharmaceutically acceptable salt thereof, so as to kill the cells.

9. The medicament according to claim 6, wherein the wavelength effective for activating the glycosylated chlorin derivative or a pharmaceutically acceptable salt thereof is 600 to 800 nm.
